(19) United States
(12) Patent Application Publication
(10) Pub. No.: US 2010/0160342 A1
(43) Pub. Date: Jun. 24, 2010

(54) Compositions of Topical Ocular Solutions to Deliver Effective Concentrations of Active Agent to the Posterior Segment of the Eye

(75) Inventors: Masood A. Chowhan, Arlington, TX (US); Wesley Wehsin Han, Arlington, TX (US); L. Wayne Schneider, Crowley, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/633,322

(22) Filed: Dec. 8, 2009

Related U.S. Application Data
(60) Provisional application No. 61/139,701, filed on Dec. 22, 2008.

(51) Int. Cl. A61K 31/496; A61P 27/02

(52) U.S. Cl. 514/252.19

(57) ABSTRACT

The present invention relates to development of efficacious pharmaceutical compositions comprising an active agent, such as the free base or hydrochloride salt of tandospirone, for topical delivery to the eye for the treatment of retinal disorders.

This application claims priority under 35 U.S.C. §119 to U.S. Provisional Patent Application No. 61/139,701 filed Dec. 22, 2008, the entire contents of which are incorporated herein by reference.

### BACKGROUND OF THE INVENTION

1. Field of the Invention

The present invention relates to unique pharmaceutical compositions for topical ocular administration containing tandospirone. Such compositions are useful for providing tandospirone to the posterior segment of the eye for the treatment of disorders affecting such tissues.

2. Description of the Related Art

There is no cure for the diseases caused by ocular neovascularization and enhanced vascular permeability. The current treatment procedures of AMD include laser photocoagulation and photodynamic therapy (PDT). The effects of photocoagulation on ocular neovascularization and increased vascular permeability are achieved only through the thermal destruction of retinal cells. PDT usually requires a slow infusion of the dye, followed by application of non-thermal laser-light. Treatment usually causes the abnormal vessels to temporarily stop or decrease their leaking PDT treatment may have to be repeated every three months up to 3 to 4 times during the first year. Potential problems associated with PDT treatment include headaches, blurring, and decreased sharpness and gaps in vision and, in 1-4% of patients, a substantial decrease in vision with partial recovery in many patients. Moreover, immediately following PDT treatment, patients must avoid direct sunlight for 5 days to avoid sunburn. Recently, a recombinant humanized IgG monoclonal antibody fragment (ranibizumab) was approved in the US for treatment of patients with age-related macular degeneration. This drug is typically administered via intravitreal injection once a month. There is currently no approved agent for treatment of disorders involving the tissues at the back of the eye that may be administered topically.

Mechanistically, for a drug molecule to reach the posterior segment of the eye, the active molecule must be penetrated through the cornea and then diffused through the anterior chamber, iris, lens, and vitreous body. It is a very long and tortuous pathway. The resultant bioavailability is poor. Alternatively, it has been proposed that the active molecule can be delivered to the posterior segment of the eye by first penetrating through the conjunctiva-sclera membrane/tissue and then the drug could be diffused along the sclera tissue and the eye globe to reach the back of the eye via vasculature. One embodiment of the present invention provides a set of pharmaceutical requirements from which the active molecule can be effectively delivered to the posterior segment of the eye, through the conjunctiva-sclera pathway.

Many compounds that may be considered potentially useful in treating ocular neovascularization and enhanced vascular permeability-related and other disorders, are poorly soluble in water. A poorly water soluble compound is a substance that is not soluble at a therapeutically effective concentration in an aqueous physiologically acceptable vehicle. Aqueous solubility is an important parameter in formulation development of a poorly water soluble compound.

Tandospirone is an anxiolytic agent developed and marketed by Sumitomo in Japan. The citrate salt of tandospirone is administered in tablet form for treatment of this indication. The tandospirone base has a molecular weight of 383.75 and the citrate ion has a molecular weight of 192.07. Thus, the citrate ion comprises approximately one-third (⅓) of the molecular weight of tandospirone citrate. Nevertheless, tandospirone citrate, in oral dosage form, has demonstrated sufficient bioavailability for achieving the efficacy for the anxiolytic product.

Formulating the citrate salt of tandospirone into a topical ocular formulation carries with it some inherent difficulties, however. Unlike an oral dosage form, a topical ocular formulation has some unique requirements. The active agent must not only be bioavailable, but the formulation must be comfortable to the patient. For example, it should not cause ocular stinging upon topical administration to the eye. The drawbacks of an uncomfortable eye drop are numerous. Due to the ocular stinging, patient compliance is likely to decrease. The stinging also often causes excessive tearing, resulting in a reduction in bioavailability of the active agent as the tear washes the agent away.

What is needed is a formulation that provides increased solubility of the compound while also providing sufficient bioavailability of the compound via topical administration so as to maintain its therapeutic potential.

The present invention provides safe and effective formulations for topical ocular administration of poorly soluble compounds for the treatment of ocular diseases affecting the tissues at the back of the eye, such as those ocular disorders caused by endothelial cell proliferation, vascular leakage, inflammation and angiogenesis. The invention further provides formulations and methods for administering such formulations along the conjunctiva-sclera pathway.

### SUMMARY OF THE INVENTION

The present invention overcomes these and other drawbacks of the prior art by providing ocular compositions for treating ocular diseases due to angiogenesis, enhanced endothelial cell proliferation, inflammation, or increased vascular permeability. Within one aspect of the present invention, a pharmaceutical composition is provided wherein the free base form of tandospirone or the hydrochloride salt of tandospirone is incorporated into an ophthalmic solution for topical delivery to the eye of a patient. Topical application of the compositions of the present invention delivers therapeutic levels of the active agent to the posterior segment (i.e., retina, choroid, etc.) of the eye. In another aspect the invention provides a set of pharmaceutical requirements from which the active molecule, tandospirone, can be effectively delivered to the posterior segment of the eye, through the conjunctiva-sclera pathway.

The concentration of the active agent used in this present invention will generally be substantially greater than 0.01%. A concentration of 0.1% to 10 wt % is preferred, with a concentration of 0.25% to 5% being more preferred, and a concentration of 0.5% to 2.0% being most preferred. Other preferred aspects of the active agent for use in the compositions of the present invention include a pKa close to physiological pH; highly lipophilic, and; practically insoluble (i.e., less than 0.1%) at physiological pH. Preferred compositions of the invention will be a solution at pH 4.0 and above and will be non-stinging upon topical application to the eye. The buffer salt use in the compositions of the present invention will preferably have a pKa that is not in the range between the formulation pH and the physiological pH of 7.4.

Another preferred criterion for improving the bioavailability of the active agent is establishing a pH range for the composition which provides the highest solubility. It will be apparent to the skilled artisan that the pH of the composition will depend upon the concentration and the salt form of the active agent in the composition. Based on the solubility pH profile, the pH of the present invention will typically be between pH 4.5 and pH 7.0, with the preferred pH range being 4.7 to 5.5. As shown in the solubility pH profile (FIG. 1), the solubility of tandospirone citrate salt is dramatically less than that of the free base. The effect is attributable to the formation of the less soluble tandospirone citrate salt crystals. Another embodiment of the invention is the discovery of this unexpected solubility profile which is affected by the crystalline salt formation. The discovery offers yet another reason for using free base or HCl salt for the present invention.

An example of a preferred composition according to the invention includes about 1.1% (w/v) hydrochloride salt of tandospirone as the active agent; and about 0.01% (w/v) benzalkonium chloride as the buffer salt; and has a pH of about 5.1±0.4. Another preferred composition according to the invention includes from about 0.1% to 2% tandospirone (w/v) as the hydrochloride salt or the free base; and about 0.5% HPMC. The preferred composition may be in the form of a solution or a suspension.

The present invention further provides a method of treating ocular diseases of the posterior segment of the eye by topically administering the compositions described herein. For example, the compositions of the present invention are preferably administered to the eye of a patient suffering from an angiogenesis or enhanced vascular permeability related ocular disorder, or a disorder characterized by neovascularization or vascular permeability, via topical ocular administration.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1 shows the solubility of tandospirone as function of the pH of the composition.

FIG. 2 shows the pH effect of a 0.2% solution of tandospirone containing 0.5% HPMC on the concentration of tandospirone in the aqueous humor and the retina delta.

FIG. 3 shows the concentration of tandospirone in the aqueous humor and the retina delta 30 minutes post-instillation after administration of a 1% solution of tandospirone (pH 6.0) including 0.5% HPMC vs. administration of a 1% suspension of tandospirone (pH7.5) including 0.5% HPMC.

FIG. 4 shows ERG a- and b-wave response amplitudes from rats dosed topical ocular with 1.75% ophthalmic solution containing a highly insoluble active agent five (5) days after blue-light exposure.

FIG. 5 shows re-evaluation of the flash-evoked electrical response of the retina after a 1-month recovery period in rats dosed topical ocular with 1.75% ophthalmic solution containing a highly insoluble active agent. Such re-evaluation confirmed the irreversibility of this light-induced functional lesion.

### DETAILED DESCRIPTION PREFERRED EMBODIMENTS

As noted above, the present invention provides compositions that contain an active agent having poor water solubility, such as tandospirone, for use in the treatment of ocular disorders caused by endothelial cell proliferation, enhanced vascular permeability, inflammation, or angiogenesis. The compositions of the invention are useful in treating disorders associated with microvascular pathology, increased vascular permeability and intraocular neovascularization, including diabetic retinopathy (DR), age-related macular degeneration (AMD) and retinal edema.

Briefly, within the context of the present invention, an active agent should be understood to be an agent that is poorly water soluble, such as tandospirone. In general, is the drug substances that will be useful in the compositions of the invention will be highly lipophilic and practically insoluble (less than 0.1%) at physiological pH; and will have a pKa close to physiological pH. The compositions of the invention will have a required effective drug concentration that is substantially greater than 0.01%; will typically be formulated as a solution at pH 4.0 and above; will be non-stinging; and the pKa of the buffer salt in the composition will not be in the range between the formulation pH and the physiological pH of 7.4.

The present inventors have discovered that the citrate form of the compound tandospirone stings upon administration to the eye. This ocular stinging is further aggravated by the fact that the ophthalmic solution has to be formulated in acidic conditions, such as pH 5, due to the compound's solubility-pH profile. Tandospirone has two pKa's: 2.17 and 7.54. According to the Handerson-Hasselbach relationship, the solubility of tandospirone is dramatically increased as the pH decreases below its pKa of 7.54.

The present inventors found that an ophthalmic solution containing the free base form or the hydrochloride salt of tandospirone is more comfortable than a composition containing the citrate salt of tandospirone when administered topically. The precise reasons for the improved comfort are not known. However, without being bound by theory, it is postulated that the solution of a high concentration of citric acid at a low pH aggravates the sensory nerves, thus making the tandospirone sting. Furthermore, since citric acid has three pKa's (3.15, 4.77, and 6.4) its high buffer capacity will keep the pH of the tear film in an acidic condition for an extended period of time. The citric acid is neutralized by the tear film components at a very slow rate as the pH of the tear film moves upward against the pKa of 6.4. It is well known that acidic solutions alone cause ocular stinging. The tandospirone ophthalmic solution of the invention is far more comfortable and far less stinging at a pH within the ranges described herein when the free base or the hydrochloride salt of tandospirone is used instead of the citrate salt.

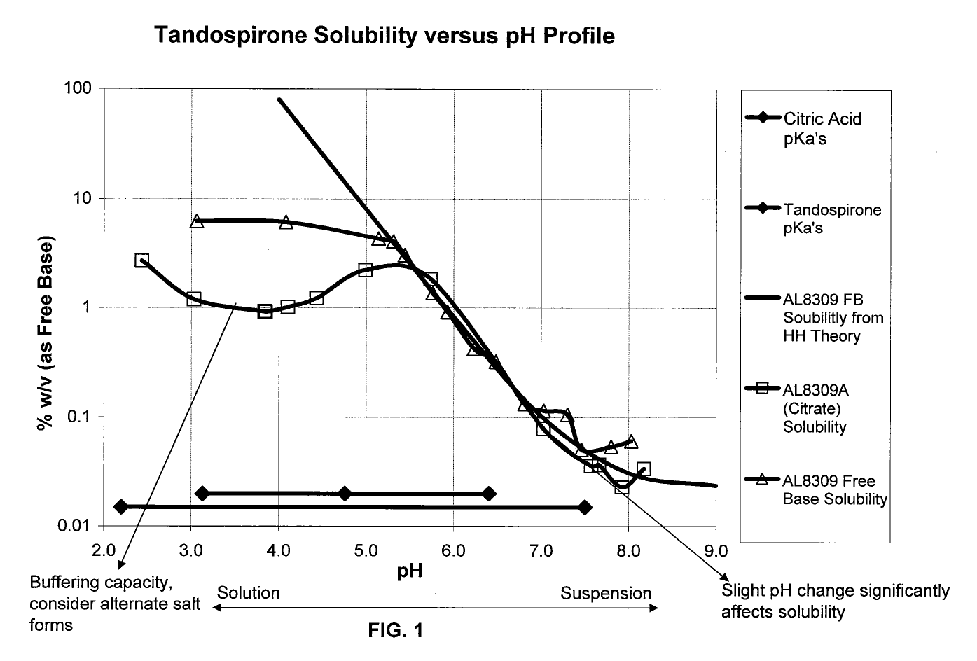

The present inventors further discovered that the solubility of tandospirone citrate salt is decreased substantially from the theoretical solubility as predicted by the Handerson-Hasselbach relationship when the pH of the solution is formulated between the 3.15 and 4.77 pKa's of citric acid. In this pH range, citric acid carries one positive charge and tandospirone carries one negative charge. The charge interaction causes precipitation of the tandospirone citrate salt, which makes the composition unstable. Therefore, the citrate salt of tandospirone is unacceptable when the target concentration of tandospirone in the ophthalmic solution is higher than that observed in the solubility-pH profile. The inventors have shown that the free base form of tandospirone does not exhibit this solubility dip observed for the citrate salt (FIG. 1)

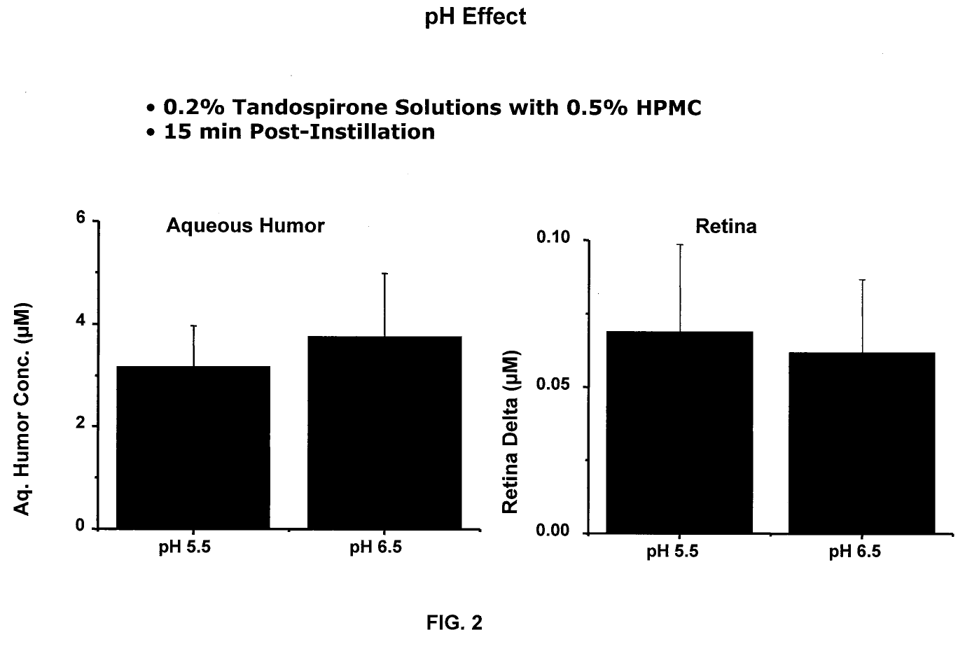

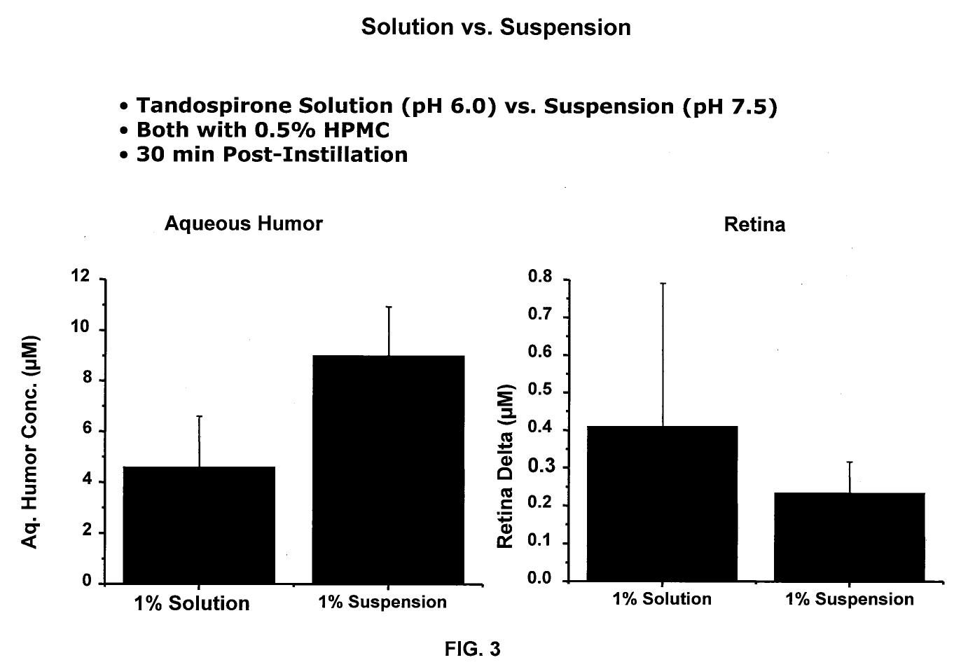

The ocular bioavailability of tandospirone indicates that conventional approaches to improve the bioavailability for the anterior segments of the eye do not work for the posterior segment of the eye. The result suggests that the penetration through the cornea pathway is not operative, which implies that the conjunctiva-sclera pathway could be the controlling factor for the bioavailability of the posterior segment of the eye. The inventors' were able to achieve unexpectedly high levels of tandospirone in the retinal tissues via topical dosing (FIG. 2 and FIG. 3). A set of pharmaceutical requirements for an ophthalmic composition that will provide such unexpectedly high drug levels in the retinal tissues is as follows:
1. The composition will contain a drug substance that is highly lipophilic and practically insoluble (less than 0.1%) at physiological pH;
2. The drug substance in the composition will have a pKa close to physiological pH;
3. The salt form of the drug substance in the composition will not have solubility less than that of the free base and hydrochloride salt crystals;
4. The effective drug concentration in the composition will be substantially greater than 0.01%; preferably greater than 0.1%; most preferably greater than 0.25%;
5. The composition will be formulated as a solution at about pH 4.0 to pH 7.0 and will be non-stinging;
6. The composition will contain a buffer salt having a pKa that is outside of the range between the formulation pH and the physiological pH of 7.4;

Compositions meeting the requirements above will deliver effective concentrations of the drug substance to the posterior segment of the eye. In preferred embodiments, the drug substance or active agent will be tandospirone, which, as used herein, includes tandospirone and any pharmaceutically acceptable salt or other form of tandospirone, other than tandospirone citrate salt. The preferred form of tandospirone for use in the compositions described herein is the free base or the hydrochloride salt. The compositions described herein are particularly desirable for topical treatment of age related macular degeneration (AMD) and AMD related maladies such as geographic atrophy secondary to wet AMD

The formulations of the present invention provide a number of advantages over conventional formulations. One advantage of the present invention is that therapeutic levels of the active drug substance contained within the composition reaches retinal tissues via topical delivery to the eye. Another advantage is that the compositions are non-stinging when delivered topically to the eye.

The compositions of the present invention may be formulated as aqueous or non-aqueous solutions, but will preferably be aqueous. Additionally, the compositions may be formulated as suspensions, gels, emulsions and other dosage forms known to those skilled in the art.

The ophthalmic compositions of the present invention will be formulated so as to be compatible with the eye and/or contact lenses to be treated with the compositions. A preferred range of osmolality for the ophthalmic compositions of the present invention is 150 to 350 milliOsmoles per kilogram (mOsm/kg). A range of 200 to 300 mOsm/kg is particularly preferred and an osmolality of about 290 mOsm/kg is most preferred. The pH for the ophthalmic compositions of the present invention can range from about 4.5 to about 7.0 but may be preferably relatively low as detailed herein. Since, often the ophthalmic formulations are required to be isotonic or near isotonic, the tonicity of the formulations can be adjusted with suitable non ionic tonicity agents including but not limited to propylene glycol, glycerin, mannitol and sorbitol.

The specific dose level of the active agent for any particular human or animal depends upon a variety of factors, including the activity of the active compound used, the age, body weight, general health, time of administration, route of administration, and the severity of the pathologic condition undergoing therapy.

The formulations described herein are intended to be delivered topically. In preferred embodiments of the present invention, the amount of active agent, or poorly water soluble agent, will be from about 0.1% to about 10%. More preferably, the amount to of active agent will be from about 0.25% to about 5%; and most preferably from about 0.5% to about 2.0%.

A general composition of an ophthalmic formulation of tandospirone hydrochloride is provided in Table 1. The general composition provided in Table 1 includes a preservative as part of the formulation.

TABLE 1
General Composition of Topical Ophthalmic Formulation

| Ingredient | Amount (w/v, %) |
|---|---|
| Tandospirone hydrochloride | 1.0-5.0 |
| Sodium Acetate (Trihydrate) | 0.1-1 |
| Sodium Chloride | q.s. to isotonic |
| Benzalkonium Chloride | 0.001-0.1 |
| Disodium Edetate, Dihydrate | 0.001-0.5 |
| Hydrochloric acid | q.s. to pH 4.0 to 7.5 |
| Sodium Hydroxide | q.s. to pH 4.0 to 7.5 |
| Water for Injection | q.s. to 100% |

Another composition of an ophthalmic formulation of tandospirone hydrochloride is provided in Table 2. The general composition provided in Table 2 does not include a preservative as part of the formulation.

TABLE 2
General Non-preserved Composition
of Topical Ophthalmic Formulation

| Ingredient | Amount (w/v, %) |
|---|---|
| Tandospirone hydrochloride | 1.0-5.0 |
| Sodium Acetate (Trihydrate) | 0.1-1 |
| Sodium Chloride | q.s. to isotonic |
| Hydrochloric acid | q.s. to pH 4.0 to 7.5 |
| Sodium Hydroxide | q.s. to pH 4.0 to 7.5 |
| Water for Injection | q.s. to 100% |

The compositions of the present invention are useful for treating disorders affecting retinal tissues. Such disorders include, but are not limited to, age-related macular degeneration, diabetic retinopathy, geographic atrophy,

In a further embodiment, the ophthalmic compositions of the invention are formulated to provide for a retinal concentration of about 0.1-100 nanomolar (nM) or, in a further embodiment, 1-10 nM. Topical compositions are delivered to the surface of the eye one to four times per day according to the routine discretion of a skilled clinician. The pH of the formulation should be between about pH 4 and about pH 9, and will preferably be between about pH 4.5 and about pH 7.4. In preferred aspects, the pH of the formulation of the invention will be lower than the pKa of the active drug molecule, in order to best improve the solubility.

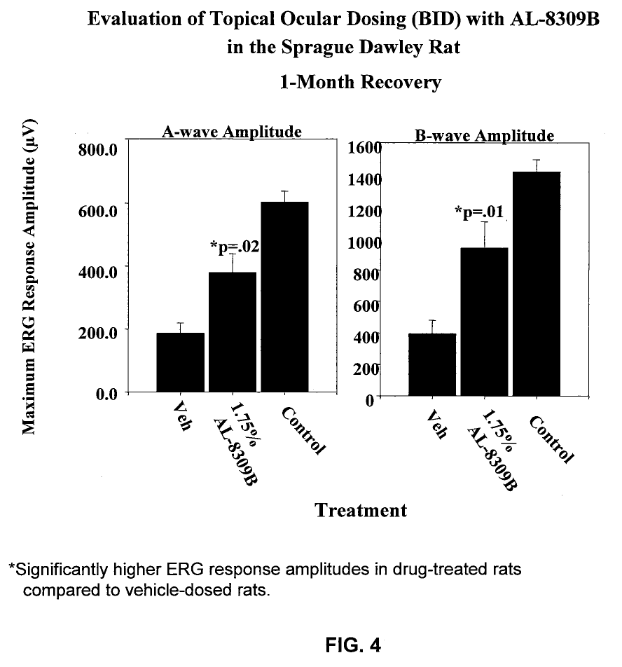

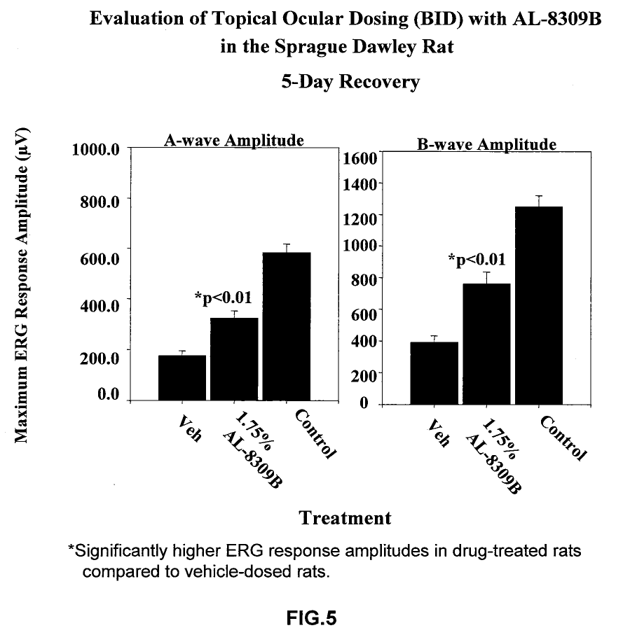

An “effective amount” refers to that amount of active agent that is able to treat retinal disorders, such as by preventing AMD, preventing damage to the retinal tissues, decreasing lesion size in the macula, decreasing or preventing geographic atrophy, decreasing photoreceptor and/or retinal pigmentation epithelial cell loss (see Example 2, FIG. 4 and FIG. 5), and delaying or preventing the onset of symptoms in a subject at risk for developing diabetic retinopathy or drusen formation in age-related macular degeneration. The effective amount of a formulation may depend on factors such as the age, race, and sex of the subject, or the severity of the retinopathy or degree of drusen formation or geographic atrophy, for example. In one embodiment, the agent is delivered topically to the eye and reaches the retina or drusen at a therapeutic dose thereby ameliorating the diabetic retinopathy, geographic atrophy, or drusen formation process.

While the precise regimen is left to the discretion of the clinician, the resulting solution or solutions are preferably administered by placing one drop of each solution(s) in each eye one to four times a day, or as directed by the clinician.

An ophthalmically acceptable carrier refers to those carriers that cause at most, little to no ocular irritation, provide suitable preservation if needed, and deliver one or more active agents as described herein in a homogenous dosage. For ophthalmic delivery, an active agent may be combined with opthalmologically acceptable preservatives, co-solvents, surfactants, viscosity enhancers, penetration enhancers, buffers, sodium chloride, or water to form an aqueous, sterile ophthalmic suspension, solution, or viscous or semi-viscous gels or other types of solid or semisolid composition such as an ointment. Ophthalmic solution formulations may be prepared by dissolving the agent in a physiologically acceptable isotonic aqueous buffer. Further, the ophthalmic solution may include an opthalmologically acceptable surfactant to assist in dissolving the agent. Viscosity building compounds, such as hydroxymethyl cellulose, hydroxyethyl cellulose, methylcellulose, polyvinylpyrrolidone, or the like, may be added to the compositions of the present invention to improve the retention of the compound.

With respect to the present invention, certain preferred embodiments of the compositions described herein will not include viscosity enhancers. The present inventors have found that viscosity enhancers, such as HPMC, did not improve the retinal drug level as it did for the aqueous humor drug level.

The effect of viscosity enhancers in a pharmacokinetics study of a formulation to containing tandospirone showed:
AqH: 0.5% HPMC ˜3-fold >0% HPMC
Retina: 0.5% HPMC ˜0% HPMC

The addition of viscosity enhancers to compositions containing tandospirone or a salt of tandospirone and having an acidic pH may cause ocular irritation and discomfort. The ocular discomfort may reduce the ocular bioavailability.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

This example illustrates the preparation of a topical formulation, according to the invention.

| Ingredient | Amount (w/v, %) |
|---|---|
| Tandospirone hydrochloride | 1.925 |
| Sodium Acetate (Trihydrate) | 0.14 |
| Sodium Chloride | 0.54 |
| Benzalkonium Chloride | 0.01 |
| Disodium Edetate, Dihydrate | 0.01 |
| Hydrochloric acid | q.s. to pH 5.1 ± 0.2 |
| Sodium Hydroxide | q.s. to pH 5.1 ± 0.2 |
| Water for Injection | q.s. to 100% |

### Preparation of the Formulation

In a suitable vessel, weigh and add sodium acetate, sodium chloride and Disodium Edetate, dehydrate. Add Tandospirone Hydrochloride raw material to the vessel. Then proper amount of water for injection is added to the vessel and mixed thoroughly until all ingredients are dissolved in the solution. The pH of the solution may be to be adjusted to around 5.0 to facilitate the dissolving. Benzalkonium chloride then is added and pH of the solution is adjusted properly.

### Example 2

### Evaluation of 1.75% Topical Ocular Formulation (BID) Containing a Highly Insoluble Active Agent in the Rat Photo-Oxidative Induced Retinopathy Model

Summary. Sprague Dawley rats were dosed topical ocular (OU, BID) starting 21 days prior to light exposure. Five days after light exposure, retinal function was assessed in rats dosed with 1.75% Topical Ocular Formulation (BID) containing a highly insoluble active agent and ERG a- and b-wave response amplitudes were greater than 2-fold higher than response amplitudes measured in vehicle-dosed rats. Significant protection (P<0.05) of ERG response amplitudes was also measured in rats after a 1-month recovery period.

Methods

Subjects and Dosing: Male Sprague Dawley albino rats were assigned to experimental groups which received either vehicle or 1.75% Topical Ocular Formulation (BID) containing a highly insoluble active agent. Rats were dosed topical ocular (BID) starting 21 days prior to light exposure, once immediately before the light exposure began and post-dosed for two days after light exposure. Control rats (N=6) were housed in their home cage under normal cyclic-light exposure.

Induction of Photochemical Lesions: Photo-oxidative induced lesions were generated by exposure to blue light (3.1×103 mW/cm2, =450 nm, half-amplitude bandpass=425-475 nm) for 6 hours.

Electrodiagnostic Evaluation: Flash ERGs were recorded from a platinum-iridium wire loop electrode positioned on the cornea and were elicited by viewing a ganzfeld. Electrical responses to a series of light flashes increasing in intensity were digitized to analyze temporal characteristics of the waveform and response voltage-log intensity (VlogI) relationship. The amplitude of the a-wave was measured as the voltage difference between the average of the 10-ms baseline recorded prior to the flash and the trough of the a-wave. The b-wave was measured as the voltage difference between the peak of the b-wave and trough of the a-wave.

Results

5-day Recovery: Blue-light exposure to vehicle-dosed rats resulted in a significant reduction in retinal function (t-test, P<0.002), a-wave response amplitudes were reduced 74% and b-wave response amplitudes were reduced 75% (FIG. 4). Five days after blue-light exposure, ERG a- and b-wave response amplitudes from rats dosed topical ocular with 1.75% Topical Ocular Formulation (BID) containing the active agent were greater than 2-fold higher and were statistically different (P<0.01) than response amplitudes measured in vehicle-dosed rats. In rats dosed with 1.75% Topical Ocular Formulation (BID) containing the highly insoluble active agent, the maximum ERG a-wave response amplitude was 352 μV (SEM±53 μV) and the maximum b-wave amplitude was 795 μV (SEM±129 μV) after a 5-day recovery.

1-month Recovery: Re-evaluation of the flash-evoked electrical response of the retina after a 1-month recovery period confirmed the irreversibility of this light-induced functional lesion (FIG. 5). ERG a- and b-wave response amplitudes were reduced 69% and 72% in vehicle-dosed rats, respectively. ERG response amplitudes recorded from albino rats dosed topical ocular with 1.75% Topical Ocular Formulation (BID) containing the active agent were significantly higher (P<0.02) compared to responses measured in vehicle-dosed rats. The maximum ERG a-wave response amplitude was 380 μV (SEM±59 μV) and the maximum b-wave response amplitude was 937 μV (SEM±166 μV) from rats dosed with the 1.75% Topical Ocular Formulation (BID) containing the active agent.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

All references mentioned herein are specifically incorporated herein by reference.

We claim:

1. A topical ophthalmic composition, said composition comprising:
an active agent having a lipophilicity measured by the distribution coefficient, Log D of greater than 1.4, a solubility at physiological pH less than 0.1%, and a pKa between 7.0 and 8.0, wherein the effective concentration of the active agent in the composition is greater than 0.1%; and
a buffer salt having a pKa outside the range between the formulation pH and the physiological pH of 7.4; and a buffer salt that does not form salt crystal that is less soluble than that of the free base;
wherein the composition is a solution containing no surface active agent;
wherein the composition is a solution having a pH range between 4.0 and 7.0.

2. The ophthalmic composition of claim 1, wherein the active agent is tandospirone or a salt thereof, provided that the active agent is not tandospirone citrate salt.

3. The ophthalmic composition of claim 2, wherein the crystalline form of tamdospirone is selected from the group consisting of tandospirone free base and tandospirone hydrochloride salt.

4. The ophthalmic composition of claim 1, wherein the concentration of the active agent is from 0.1% to 10%.

5. The ophthalmic composition of claim 1, wherein the said concentration of the active agent is from 0.1% to 10%.

6. The ophthalmic composition of claim 1, wherein the composition has a pH range between 4.7 and 5.5.

7. The ophthalmic composition of claim 1, wherein the pH of the composition is about 5.1.

8. The ophthalmic composition of claim 1, wherein the composition does not cause a stinging sensation upon topical administration to the eye.

9. A method for treating geographic atrophy in a patient suffering therefrom, said method comprising topically administering to the eye of said patient an ophthalmic composition of claim 1.
